(19) United States
(12) Patent Application Publication
(10) Pub. No.: US 2016/0289769 A1
(43) Pub. Date: Oct. 6, 2016

(54) Methods for Combining Single Cell Profiling with Combinatorial Nanoparticle Conjugate Library Screening and In Vivo Diagnostic System

(71) Applicant: Verily Life Sciences LLC, Mountain View, CA (US)

(72) Inventors: Jerrod Joseph SCHWARTZ, Mountain View, CA (US); Alberto Clemente VITARI, San Francisco, CA (US)

(73) Assignee: Verily Life Sciences LLC

(21) Appl. No.: 15/084,877

(22) Filed: Mar. 30, 2016

Related U.S. Application Data
(60) Provisional application No. 62/140,308, filed on Mar. 30, 2015.

(51) Int. Cl. C12Q 1/68; C12N 15/10; G01N 15/14

(52) U.S. Cl. CPC C12Q 1/6886; G01N 15/14; C12N 15/1065; G01N 2015/149; G01N 2015/1006

(57) ABSTRACT

Methods for characterizing a cell employ a library of nanoparticle conjugates of one or more types, each type of nanoparticle conjugate comprising a nanoparticle, targeting entities of one or more types, and tags of one or more types, to determine the genotype and phenotype of a cell as well as other characteristics. The tags can include oligonucleotide barcodes.

### CROSS-REFERENCE

This application claims the benefit of priority from U.S. Provisional Application No. 62/140,308, filed Mar. 30, 2015, which is incorporated by reference in its entirety.

### BACKGROUND

Unless otherwise indicated herein, the materials described in this section are not prior art to the claims in this application and are not admitted to be prior art by inclusion in this section.

A number of diagnostic methods have been developed to evaluate physiological conditions of a person by detecting and/or measuring one or more analytes in a person's blood or other bodily fluids or tissue. One or more target analytes could be any analytes that, when present in or absent from the blood, or present at a particular concentration or range of concentrations, may be indicative of a medical condition or health state of the person. These target analytes could include tumor cells released from a primary tumor and circulating in the blood stream. While many of the diagnostic methods can be used to detect cancer, it is difficult to detect rare cells, particularly tumor cells, that are present in miniscule numbers in the blood stream.

It is known that solid cancer tumors include genetically and phenotypically heterogeneous populations of cells due to a high rate of somatic mutation, clonal expansion and diverse tumor microenvironment. This genetic and/or phenotypic diversity manifests itself as cells are released from the primary tumor and begin to transit the circulatory system as circulating tumor cells. While much effort has been devoted into developing cell profiling techniques for characterizing the genotype and phenotype of single cells, including tumor cells, additional information regarding the tumor is needed to further characterize the tumor cell so that the medical condition of a patient can be more accurately determined and monitored and the patient can receive effective treatment.

### SUMMARY

One aspect of the present disclosure provides a method for characterizing a cell, e.g., a tumor cell. The method includes: (a) contacting cells with nanoparticle conjugates of one or more types to form labeled cells, each type of nanoparticle conjugate comprising a nanoparticle, targeting entities of one or more types, and tags of one or more types; (b) partitioning the labeled cells into separate partitions; (c) lysing each labeled cell to release nucleic acid molecules, each nucleic molecule having a first end and a second end; (d) adding partition specific adaptors and common adaptors to each partition, wherein each partition specific adaptor comprises an oligonucleotide barcode, wherein the partition specific adaptors are different for each partition, wherein each common adaptor comprises an oligonucleotide of a predetermined sequence and wherein the common adaptors are the same for each partition; (e) ligating the partition specific adaptor to a first end of each nucleic acid molecule and the common adaptor to a second end of each nucleic acid molecule to form modified nucleic acid molecules; (f) ligating the partition specific adaptor to the nanoparticle conjugate to form a partition specific adaptor modified nanoparticle conjugate; (g) amplifying the modified nucleic acid molecules and the partition specific adaptor of the partition specific adaptor modified nanoparticle conjugates; (h) pooling the amplified modified nucleic acid molecules and the amplified partition specific adaptors from the partition specific adaptor modified nanoparticle conjugates from each partition to form a library; and (i) sequencing the library. In one embodiment, the nucleic acid molecules comprise genomic DNA molecules and mRNA molecules. In another embodiment, prior to step (d), step (C1) is performed which entails reverse transcribing the mRNA molecules to form complementary DNA molecules (cDNA). In some embodiments, step (g) amplifying involves a polymerase chain reaction (PCR). In some embodiments, step (i) sequencing employs a massively parallel sequencer. In some embodiments, the separate partitions are contained in separate wells. In other embodiments, the tags of one or more types comprise an oligonucleotide barcodes of one or more types.

In another aspect, the present disclosure provides a method. The method involves (a) contacting cells with nanoparticle conjugates of one or more types to form labeled cells, each type of nanoparticle conjugate comprising a nanoparticle, targeting entities of one or more types, and a plurality of first oligonucleotide barcodes; (b) partitioning and immobilizing the labeled cells into spatially discrete regions on a support, each labeled cell comprising mRNA molecules; (c) sequencing the first oligonucleotide barcodes of each labeled cell; (d) reverse transcribing the mRNA molecules of each labeled cell into cDNA molecules to form a library for each labeled cell; and (e) sequencing the library for each labeled cell. In one embodiment, steps (c) and (e) sequencing involves fluorescent in situ sequencing. In other embodiment, the support comprises a glass slide coated with a gel matrix.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A illustrates an example nanoparticle conjugate including a nanoparticle, a targeting entity, and an oligonucleotide barcode.

FIG. 1B illustrates a flow diagram for an example method for characterizing a cell. Single cells are contacted with a library of nanoparticle conjugates of several types to form tagged cells which are bound to one or more types of nanoparticle conjugates. Each tagged cell can be profiled by any suitable method such as fluorescent in situ sequencing (FISSEQ), RNA sequencing (RNAseq), exome capture, genome sequencing. The nanoparticle conjugates can also be sequenced in order to identify the specific nanoparticle conjugates that are bound to the cell.

FIG. 2 illustrates an example method for characterizing a cell. Single cells are contacted with a library of nanoparticle conjugates of several types to form tagged cells which are bound to one or more types of nanoparticle conjugates, each type of nanoparticle conjugates having a unique oligonucleotide barcode or tag. Each tagged cell is then partitioned into separate wells in a microtiter plate (FIG. 2A). FIG. 2B shows that the partitioned cells are lysed to release their nucleic acid content. Partition specific adaptors and common adaptors are then added to the lysed partitioned cells (FIG. 2C). The adaptors are ligated to the nucleic acids and the resulting modified nucleic acids are then amplified (FIG. 2D). The amplified modified nucleic acids from each well is then pooled (FIG. 2E) and the pooled mixture is then converted into a library which is then sequenced.

FIG. 3 illustrates an example method for characterizing a cell. Single cells are contacted with a library of nanoparticle conjugates of several types to form tagged cells which are bound to one or more types of nanoparticle conjugates, each type of nanoparticle conjugates having a unique oligonucleotide barcode or tag. Each tagged cell is then partitioned and immobilized onto spatially discrete areas on a glass microscope slide (FIG. 3A). The tags of the nanoparticle conjugates of each partition are separately sequenced (FIG. 3B). The nucleic acid content of each partitioned cell are separately sequenced (FIG. 3C).

FIG. 4A illustrates an example nanoparticle tag amplification using a partition specific adaptor (PCR primer) 410 and a common adaptor (PCR primer) 420 to perform PCR amplication of nanoparticle barcodes 430.

FIG. 4B illustrates an example DNA amplification of genomic DNA 400, the whole genome amplification (WGA) using a partition-specific primer 410 followed by amplification with common primers to produce amplified genomic DNA 450.

FIG. 4C illustrates an example whole genome amplification (WGA) of genomic DNA 400 using with a common set of primers 460 and partition-specific primers.

FIG. 4D illustrates two example paths for amplification of mRNA 480 via reverse transcription with partition specific adaptors 485 to produce cDNA molecules 490 and 495.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. NANOPARTICLE CONJUGATES AND LIBRARY OF NANOPARTICLE CONJUGATES

The present disclosure provides methods and a system that employ nanoparticle conjugates and libraries of such conjugates for characterizing cells, particularly tumor cells, as well as the detection of target analytes such as rare cells in vitro or in vivo. Functionalized nanoparticles are important materials for biological applications, particularly for sensing, separation, and imaging. To achieve target specificity, nanoparticles conjugates are prepared by conjugating nanoparticles with targeting entities and unique tags or identifiers such as oligonucleotide barcodes. The targeting entities can include any substance that can specifically bind to a marker on the surface of a cell, e.g. tumor cell, and include small molecules, proteins, antibodies, etc. The targeting entities can be bound to the nanoparticles directly or through a linker. Alternatively, the targeting entities can be bound to at least a portion of the oligonucleotide barcodes which are bound to the nanoparticle. The nanoparticle conjugates can be used to create libraries of nanoparticle conjugates of one or more types, each type of nanoparticle conjugate having unique oligonucleotide barcodes that serves as a tag or identifier of the nanoparticle conjugate. By exposing a tumor cell or collection of circulating tumor cells to a complex library of nanoparticle conjugates to tag the cells, then characterizing the genotype and phenotype of the tagged cells and the nanoparticle conjugates by high-throughput sequencing methods, it could be possible to further characterize the surface cancer markers at the single cell level.

### A. Nanoparticles

In general, nanoparticles contemplated include any compound or substance with a high loading capacity for an oligonucleotide as described herein, including for example and without limitation, a metal, a semiconductor, and an insulator particle composition, and a dendrimer (organic versus inorganic). The term “nanoparticle” refers to any particle having a diameter of less than 1000 nanometers (nm). Methods for making nanoparticles of a wide variety of materials or combination of materials are known. Representative examples of nanoparticles include, without limitation, quantum dots, plasmonic nanoparticles such as gold or silver nanoparticles, upconverting nanocrystals, iron oxide nanoparticles or other superparamagnetic or magnetic particles, silica, liposomes, micelles, carbon nanotubes, doped or undoped graphene, graphene oxide, nanodiamonds, titania, alumina, and metal oxides. In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments. Typically the nanoparticles can have a longest straight dimension (e.g., diameter) of less than 1000 nm, 900 nm, 800 nm, 700 nm, 600 nm, 500 nm, 400 nm, 300 nm, 200 nm or less. In some embodiments, the nanoparticles can have a diameter of 200 nm or less. In other embodiments, the nanoparticles have a diameter of 100 nm or less. Smaller nanoparticles, e.g. having diameters of 50 nm or less, e.g., 5 nm-30 nm, are used in some embodiments.

In one embodiment, nanoparticles are quantum dots, i.e., bright, fluorescent nanocrystals with physical dimensions small enough such that the effect of quantum confinement gives rise to unique optical and electronic properties. In certain embodiments, optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys and/or oxides thereof. In some embodiments, magnetic nanoparticles are of use in accordance with the present disclosure. “Magnetic nanoparticles” refers to magnetically responsive nanoparticles that contain one or more metals or oxides or hydroxides thereof.

In other embodiments, the nanoparticles are made from polymers or lipids See for instance, EP 2644 192; U.S. Pat. No. 8,246,968; U.S. Patent Publication No. 2013/0037977; U.S. Pat. No. 5,478,860; U.S. Patent Publ. No. 2004/0142025; International Patent Publication Nos. WO 01/05373, 2014/057432, and 2014/037498; and EP 2698066, which are incorporated by reference in their entirety.

In other embodiments, the nanoparticle comprises a bulk material that is not intrinsically fluorescent, luminescent, plasmon resonant, or magnetic. The nanoparticle comprises one or more fluorescent, luminescent, or magnetic moieties. For example, the nanoparticle may comprise QDs, fluorescent or luminescent organic molecules, or smaller nanoparticles of a magnetic material. In other embodiments, the nanoparticles are made from polymers.

In some embodiments, a nanoparticle composed in part or in whole of an organic polymer is used. A wide variety of organic polymers and methods for forming nanoparticles therefrom are known in the art. For example, nanoparticles composed at least in part of polymethylmethacrylate, polyacrylamide, poly(vinyl chloride), carboxylated poly(vinyl chloride), or poly(vinyl chloride-co-vinyl acetate-co-vinyl alcohol) may be used. Optionally the nanoparticle comprises one or more plasticizers or additives. Co-polymers, block co-polymers, and/or grafted co-polymers can be used.

In some embodiments, the nanoparticles can be labeled with any suitable reporter label including, without limitation, fluorescent and luminescent moieties such as a variety of different organic or inorganic small molecules commonly referred to as “dyes”, “labels”, or “indicators”. Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g. Valeur, B., “Molecular Fluorescence: Principles and Applications”, John Wiley and Sons, 2002; Handbook of Fluorescent Probes and Research Products, Molecular Probes, 9th edition, 2002; and The Handbook-A Guide to Fluorescent Probes and Labeling Technologies, Invitrogen, 10^{th} edition), all incorporated by reference in their entirety. In some embodiments, the labels can include non-fluorescent dyes or nanoparticles that can act as quenchers for fluorophores. Such nanoparticle labels may quench dynamically by distance modulation or molecular structure modulation in response to a change in the local environment or molecular recognition event.

In some embodiments, the nanoparticles can be biocompatible and/or biodegradable. As used herein, the term “biocompatible” refers to substances that are not toxic to cells or are present in levels that are not toxic to cells. In some embodiments, a substance is considered to be “biocompatible” if its addition to cells in vivo does not induce inflammation and/or other adverse effects in vivo. In other embodiments, the materials composing the nanoparticles can be generally recognized as safe (GRAS) or FDA-approved materials. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death. In general, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

In some embodiments, a nanoparticle which is biocompatible and/or biodegradable may be associated with a targeting entity and/or an agent to be delivered that is not biocompatible, is not biodegradable, or is neither biocompatible nor biodegradable. In some embodiments, a nanoparticle which is biocompatible and/or biodegradable may be associated with agent to be delivered is also biocompatible and/or biodegradable.

Nanoparticles can have a coating layer. Use of a biocompatible coating layer can be advantageous, e.g., if the nanoparticles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, and other nanoparticles, etc. Coatings may be applied or assembled in a variety of ways such as by dipping, using a layer-by-layer technique, self-assembly, conjugation, etc.

In some embodiments, the nanoparticles may optionally comprise one or more dispersion media, surfactants, release-retarding ingredients, or other pharmaceutically acceptable excipient. In some embodiments, nanoparticles may optionally comprise one or more plasticizers or additives.

In some embodiments, nanoparticles may be intrinsically magnetic nanoparticles. In some embodiments, fluorescent or luminescent nanoparticles, nanoparticles that comprise fluorescent or luminescent moieties, and plasmon resonant nanoparticles can be useful. In some embodiments, the nanoparticles have detectable optical and/or magnetic properties. In one embodiment, an optically detectable nanoparticle is one that can be detected within a living cell using optical means compatible with cell viability. In another embodiment, an optically detectable nanoparticle is one that can be detected within a living cell using optical means compatible with cell viability in a biological setting and that do not permanently compromise the integrity or viability of the cells or tissues. Optical detection is accomplished by detecting the scattering, emission, and/or absorption of light that falls within the optical region of the spectrum, i.e., that portion of the spectrum extending from approximately 400 nm to several microns. Optionally a sample containing cells is exposed to a source of electromagnetic energy. In some embodiments, absorption of electromagnetic energy (e.g. light of a given wavelength) by the nanoparticle or a component thereof is followed by the emission of light at longer wavelengths, and the emitted light is detected. In some embodiments, scattering of light by the nanoparticles is detected. In certain embodiments, light falling within the visible portion of the electromagnetic spectrum, i.e., the portion of the spectrum that is detectable by the human eye (approximately 400 nm to approximately 700 nm) is detected. In some embodiments, light that falls within the infrared or ultraviolet region of the spectrum is detected.

The optical property can be a feature of an absorption, emission, or scattering spectrum or a change in a feature of an absorption, emission, or scattering spectrum. The optical property can be a visually detectable feature such as, for example, color, apparent size, or visibility (i.e. simply whether or not the particle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the nanoparticles; thus polarization dependence can be measured. Features associated with hyper-Rayleigh scattering can be measured. Fluorescence detection can include detection of fluorescence modes. Luminescence detection can also be useful for optical imaging purposes. Raman scattering can also be useful as well.

In various embodiments, intrinsically fluorescent or luminescent nanoparticles, nanoparticles that comprise fluorescent or luminescent moieties, plasmon resonant nanoparticles, and magnetic nanoparticles are among the detectable nanoparticles that can be used. Such nanoparticles can have a variety of different shapes including variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (nanoparticles having four leg-like appendages), triangles, prisms, etc. Nanoparticles can be also solid or hollow and can comprise one or more layers (e.g., nanoshells, nanorings, etc.). Nanoparticles may have a core/shell structure, wherein the core(s) and shell(s) can be made of different materials. Nanoparticles may comprise gradient or homogeneous alloys. Nanoparticles may be a composite made of two or more materials, of which one, more than one, or all of the materials possess magnetic properties, electrically detectable properties, and/or optically detectable properties.

### B. Oligonucleotides

The nanoparticle conjugates include a unique label or combination of labels that act as a unique “tag” or identifier of the nanoparticle conjugate. In one embodiment, a tag can be an oligonucleotide having a unique sequence, otherwise referred to herein as an “oligonucleotide barcode.” Each nanoparticle can have one or more types of oligonucleotides barcodes attached to it. As used herein, the term “oligonucleotide” refers to short single-stranded DNA or RNA molecules of 200 or less nucleobases and includes modified forms. Likewise, the term “nucleotides” as used herein is interchangeable with modified forms as discussed herein and otherwise known in the art. In certain instances, the art uses the term “nucleobase” which embraces naturally-occurring nucleotides as well as modifications of nucleotides that can be polymerized into a molecule that functions as antisense. Herein, the terms “nucleotides” and “nucleobases” are used interchangeably to embrace the same scope unless otherwise noted. Modified bases are useful in a number of instances to minimize charge effects that a negatively charged DNA backbone may have on binding affinity (e.g., nucleic acids with methylphosphonates) or to minimize nuclease sensitivity (e.g., XNA or other nucleotide analogs). Methods of making oligonucleotides of predetermined sequences are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991), incorporated by reference in its entirety. Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

Any suitable length of oligonucleotides can be used to prepare the nanoparticle conjugates. In some embodiments, the oligonucleotide which modified the surface of a nanoparticle can be about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, or about 5 to about 10 nucleotides in length and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated. In some embodiments, oligonucleotides comprise from about 8 to about 80 nucleotides (i.e. from about 8 to about 80 linked nucleosides). One of ordinary skill in the art will appreciate that methods utilize compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide in length. In some embodiments, an oligonucleotide is a DNA oligonucleotide, an RNA oligonucleotide, or a modified form of either a DNA oligonucleotide or an RNA oligonucleotide.

The nanoparticles, the oligonucleotides or both are functionalized in order to attach the oligonucleotides to the nanoparticles to form nanoparticle conjugates. Methods for functionalizing nanoparticles and oligonucleotides are known in the art. With respect to the oligonucleotides, any suitable means for binding them to the nanoparticles can be used. Regardless of the means by which the oligonucleotides are attached to the nanoparticle, attachment in various aspects is effected through a 5′ linkage, a 3′ linkage, some type of internal linkage, or any combination of these attachments. For instance, oligonucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995), incorporated by reference in its entirety. See also, Mucic et al. Chem. Commun. 555-557 (1996), incorporated by reference in its entirety, describes a method of attaching 3′ thiol DNA to flat gold surfaces; this method can be used to attach oligonucleotides to nanoparticles. The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881, incorporated by reference in its entirety, for the binding of oligonucleotide-phosphorothioates to gold surfaces), substituted alkylsiloxanes (see, e.g. Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes, all incorporated by reference in their entirety). Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lee et al., J. Phys. Chem., 92, 2597 (1988) (rigid phosphates on metals), all incorporated by reference in its entirety.

U.S. patent application Ser. Nos. 09/760,500 and 09/820,279 and International Application Nos. PCT/US01/01190 and PCT/US01/10071 (all incorporated by reference in their entirety) describe oligonucleotides functionalized with a cyclic disulfide. The cyclic disulfides in certain aspects have 5 or 6 atoms in their rings, including the two sulfur atoms. Suitable cyclic disulfides are available commercially or are synthesized by known procedures. Functionalization with the reduced forms of the cyclic disulfides is also contemplated.

In certain aspect, nanoparticle conjugates are contemplated which include those wherein an oligonucleotide is attached to the nanoparticle through a spacer. “Spacer” as used herein means a moiety which serves to increase distance between the nanoparticle and the functional oligonucleotide, or to increase distance between individual oligonucleotides when attached to the nanoparticle in multiple copies. Thus, spacers are contemplated being located between individual oligonucleotide in tandem, whether the oligonucleotides have the same sequence or have different sequences. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, or combinations thereof.

In certain aspects, the spacer has a moiety covalently bound to it, the moiety comprising a functional group which can bind to the nanoparticles. These are the same moieties and functional groups as described above. As a result of the binding of the spacer to the nanoparticles, the oligonucleotide is spaced away from the surface of the nanoparticles and is more accessible for hybridization with its target. In instances wherein the spacer is a polynucleotide, the length of the spacer in various embodiments at least about 10 nucleotides, 10-30 nucleotides, or even greater than 30 nucleotides. The spacer may have any sequence which does not interfere with the ability of the oligonucleotides to become bound to the nanoparticles and act as a tag or barcode. The spacers should not have sequences complementary to each other or to that of the oligonucleotides. In certain aspects, the bases of the polynucleotide spacer are all adenines, all thymines, all cytidines, all guanines, all uracils, or all some other modified base.

In another embodiment, a non-nucleotide linker of the present disclosure comprises a basic nucleotide, polyether, polyamine, polyamide, peptide, carbohydrate, lipid, polyhydrocarbon, or other polymeric compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, the disclosures of which are all incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the C1 position of the sugar.

In various aspects, linkers contemplated include linear polymers (e.g., polyethylene glycol, polylysine, dextran, etc.), branched-chain polymers (see, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993) (all incorporated by reference in their entirety); lipids; cholesterol groups (such as a steroid); or carbohydrates or oligosaccharides. Other linkers include one or more water soluble polymer attachments such as polyoxyethylene glycol, or polypropylene glycol as described U.S. Pat. Nos: 4,640,835, 4,496,689, 4,301,144, 4,670,417, 4,791,192 and 4,179,337 (all incorporated by reference in their entirety). Other useful polymers as linkers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

In still other embodiments, oligonucleotide such as poly-A or hydrophilic or amphiphilic polymers are contemplated as linkers, including, for example, amphiphiles (including oligonucletides).

In one embodiment, a plurality of oligonucleotides may be attached to the nanoparticle. In one embodiment, the plurality of oligonucleotides bound to the nanoparticle can be homogenous or identical. In other embodiments, the plurality of oligonucleotides may include two or more types of oligonucleotides, and each type of oligonucleotide may be different. The plurality of oligonucleotides can include about 10 to about 100,000 oligonucleotides, about 10 to about 90,000 oligonucleotides, about 10 to about 80,000 oligonucleotides, about 10 to about 70,000 oligonucleotides, about 10 to about 60,000 oligonucleotides, 10 to about 50,000 oligonucleotides, 10 to about 40,000 oligonucleotides, about 10 to about 30,000 oligonucleotides, about 10 to about 20,000 oligonucleotides, about 10 to about 10,000 oligonucleotides, and all numbers of oligonucleotides intermediate to those specifically disclosed to the extent that the oligonucleotide-modified nanoparticle is able to achieve the desired result.

In another embodiment, the oligonucleotide is bound to the nanoparticle at a surface density of at least 10 pmol/cm², at least 15 pmol/cm², at least 20 pmol/cm², at least 10 pmol/cm², at least 25 pmol/cm², at least 30 pmol/cm², at least 35 pmol/cm², at least 40 pmol/cm², at least 45 pmol/cm², at least 50 pmol/cm², at least 55 pmol/cm², at least 60 pmol/cm², at least 65 pmol/cm², at least 70 pmol/cm², or at least 75 pmol/cm².

### C. Targeting Entity

In addition to oligonucleotides, the nanoparticle conjugate can include one or more targeting entities. The targeting entity can be directly attached to the nanoparticle with or without a linker. Alternatively, in general, a “targeting entity” is any entity that binds to a component (also referred to as a “target” or a “marker”) associated with a bodily fluid such as blood, an organ, tissue, cell, subcellular locale, and/or extracellular matrix component. In one embodiment, the targeting entity binds to a marker on the surface of a cell, e.g., tumor cell. A targeting entity may be an antibody, polypeptide, glycoprotein, carbohydrate, lipid, enzyme, nanobodies, ScFv, an ionophore, small molecule recognition element, a charge carrying small molecule, etc. A targeting entity can be a member of a specific binding pair such as a receptor-ligand or antibody-antigen. A targeting entity can be an antibody, which term is intended to include antibody fragments, characteristic portions of antibodies, single chain antibodies, etc. Synthetic binding proteins such as affibodies, etc., can be used. Peptide targeting entities can be identified, e.g., using procedures such as phage display. This widely used technique has been used to identify cell specific ligands for a variety of different cell types.

In one embodiment, the targeting entities bind to an organ, tissue, cell, extracellular matrix component, and/or intracellular compartment that is associated with a specific developmental stage or a specific disease state (i.e. a “target” or “marker”). In some embodiments, a target is an antigen or molecule on the surface of a cell, such as a cell surface receptor, an integrin, a transmembrane protein, an ion channel, and/or a membrane transport protein. In some embodiments, a target is an intracellular protein. In some embodiments, a target is a soluble protein, such as immunoglobulin. In some embodiments, a target is more prevalent, accessible, and/or abundant in a diseased locale (e.g. organ, tissue, cell, subcellular locale, and/or extracellular matrix component) than in a healthy locale.

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. In some embodiments, the moieties are attached to one another by one or more covalent bonds. In some embodiments, the moieties are attached to one another by a mechanism that involves specific (but non-covalent) binding (e.g. streptavidin/avidin interactions, antibody/antigen interactions, metal coordination, etc.). In some embodiments, a sufficient number of weaker interactions can provide sufficient stability for moieties to remain physically associated.

In one embodiment, the targeting agent is an antibody. As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” refers to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids. Antibodies to many markers are known to those of skill in the art and can be obtained commercially or readily produced by known methods such as using phage-display or yeast-display technology.

### D. Reporter Labels

In some embodiments, the nanoparticle conjugates can include one or more reporter labels (also referred to as “reporter”). In one embodiment, the reporter label can be attached to the surface of nanoparticle, the oligonucleotide barcode, or the targeting entity or in any combination of the foregoing. A “reporter label” as used herein, is intended to mean a chemical compound, molecule, ion, or particle that directly possesses or indirectly comes to possess a detectable signal. Representative examples of reporter labels include, without limitation, organic dyes, state dyes, environmentally-responsive absorbers that are sensitive to changes in oxygen, pH, and redox levels, fluorophores, phosphores, porphyrins, and conducting/responsive polymers. In some embodiments, the nanoparticle component can be labeled with one or more compounds or molecules such as fluorophores or auto-fluorescent or luminescent markers or non-optical contrast agents (e.g., acoustic impedance contrast, RF contrast and the like) or enzymes or enzyme substrates which may further assist in interrogating the nanoparticle conjugates in vivo. The labels can be used to indicate a conformational change of the targeting entity which can be indicative of target binding. The labeling moieties used in the current methods and compositions can be attached through any suitable means including chemical means, such as reduction, oxidation, conjugation, and condensation reactions. For example, any thiol-reactive group can be used to attach labeling moieties, e.g., a fluorophore, to a naturally occurring or engineered thiol group present in the targeting entity, e.g., antibody. Also, for example, reactive groups present in the targeting agent can be labeled using succinimide ester derivatives of fluorophores. See Richieri, G. V. et al., J. Biol. Chem., 267: 23495-501 (1992) which is hereby incorporated by reference.

In one embodiment, the labeling moiety can emit an optical signal. Numerous labels are known by those of skill in the art and include, but are not limited to, particles, fluorophores, haptens, enzymes and their colorimetric, fluorogenic and chemiluminescent substrates and other labels that are described in RICHARD P, HAUGLAND, MOLECULAR PROBES HANDBOOK OF FLUORESCENT PROBES AND RESEARCH PRODUCTS (9th edition, CD-ROM, (September 2002), which is herein incorporated by reference.

A fluorophore label can be any chemical moiety that exhibits an absorption maximum at or beyond 280 nm, and when covalently attached to the nanoparticle component, e.g., targeting entity, or other component retains its spectral properties. Fluorophores of the present disclosure include, without limitation; a pyrene (including any of the corresponding derivative compounds disclosed in U.S. Pat. No. 5,132,432, incorporated by reference), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine (including any corresponding compounds in U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; 6,664,047; 6,974,873 and 6,977,305; and publications WO 02/26891, WO 97/40104, WO 99/51702, WO 01/21624; EP 1 065 250 A1, incorporated by reference), a carbostyryl, a porphyrin, a salicylate, an anthranilate, an azulene, a perylene, a pyridine, a quinoline, a borapolyazaindacene (including any corresponding compounds disclosed in U.S. Pat. No. 4,774,339; 5,187,288; 5,248,782; 5,274,113; and 5,433,896, incorporated by reference), a xanthene (including any corresponding compounds disclosed in U.S. Pat. Nos. 6,162,931; 6,130,101; 6,229,055; 6,339,392; 5,451,343 and 6,716,979, incorporated by reference), an oxazine (including any corresponding compounds disclosed in U.S. Pat. No. 4,714,763, incorporated by reference) or a benzoxazine, a carbazine (including any corresponding compounds disclosed in U.S. Pat. No. 4,810,636, incorporated by reference), a phenalenone, a coumarin (including a corresponding compounds disclosed in U.S. Pat. Nos. 5,696,157; 5,459,276; 5,501,980 and 5,830,912, incorporated by reference), a benzofuran (including a corresponding compounds disclosed in U.S. Pat. Nos. 4,603,209 and 4,849,362, incorporated by reference) and benzphenalenone (including any corresponding compounds disclosed in U.S. Pat. No. 4,812,409, incorporated by reference) and derivatives thereof. As used herein, oxazines include resorufins (including any corresponding compounds disclosed in U.S. Pat. No. 5,242,805, incorporated by reference), aminooxazinones, diaminooxazines, and their benzo-substituted analogs. When the fluorophore is a xanthene, the fluorophore is optionally a fluorescein, a rhodol (including any corresponding compounds disclosed in U.S. Pat. Nos. 5,227,487 and 5,442,045, incorporated by reference), or a rhodamine (including any corresponding compounds in U.S. Pat. Nos. 5,798,276; 5,846.737 and 6,562,632, incorporated by reference). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171, incorporated by reference). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Fluorophores for use in the present disclosure include, but are not limited to, xanthene (rhodol, rhodamine, fluorescein and derivatives thereof) coumarin, cyanine, pyrene, oxazine and borapolyazaindacene. Sulfonated and/or alkylated xanthenes, fluorinated xanthenes, sulfonated coumarins, fluorinated coumarins and sulfonated cyanines can be useful. The choice of the fluorophore will determine the absorption and fluorescence emission properties of the nanoparticle. Physical properties of a fluorophore label include spectral characteristics (absorption, emission and stokes shift), fluorescence intensity, lifetime, polarization and photo-bleaching rate all of which can be used to distinguish one fluorophore from another.

Binding of the nanoparticle conjugate to a target analyte, e.g., tumor cell, may be detected with or without an interrogation signal input. The term “binding” is understood in its broadest sense to include any detectable interaction between the nanoparticle conjugate and the target analyte. For example, some nanoparticle conjugates may be functionalized with compounds or molecules, such as fluorophores or autofluorescent, luminescent or chemiluminescent markers, which generate a responsive signal with the input of a stimulus when the target binds to the nanoparticle conjugates. In other examples, the nanoparticle conjugates may produce a different responsive signal in their bound versus unbound state in response to an external stimulus, such as an electromagnetic, acoustic, optical, or mechanical energy.

In one embodiment, multiple analyte detection is possible. By immobilizing a plurality of targeting entities of different binding specificities to target analytes, each targeting entity associated directly or indirectly with distinct labels, e.g., different fluorophores, simultaneous multiple target analyte determinations can be made, thereby providing clinicians with deeper insight into the identification and assessment of health state and disease progression. The use of spectral filters and/or alternative light sources as the interrogation signal can be used to excite the label, e.g., fluorophores and detect light, e.g., fluorescent light, from the different labels, and thereby, determine the contribution of each fluorophore to the total fluorescent properties of the sample.

### E. Linkers

In one embodiment, the targeting entity, the oligonucleotides, the reporter label and/or any other component (e.g, imaging agent or drug) can be attached to the nanoparticles via a linking agent to form the nanoparticle conjugates. In other embodiments, the targeting entity and/or the reporter can be attached to each other via a linking agent. For instance, a targeting entity and/or reporter label and nanoparticle can be conjugated via a single linking agent or multiple linking agents. For example, the targeting entity and/or reporter label and nanoparticle may be conjugated via a single multifunctional (e.g., bi-, tri-, or tetra-) linking agent or a pair of complementary linking agents. In another embodiment, the targeting agent and/or reporter label and the nanoparticle are conjugated via two, three, or more linking agents. Suitable linking agents include, but are not limited to, e.g., functional groups, affinity agents, stabilizing groups, and combinations thereof

In certain embodiments the linking agent is or comprises a functional group. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some preferred embodiments, the multifunctional crosslinkers are heterobifunctional crosslinkers comprising two or more different reactive groups.

Suitable reactive groups include, but are not limited to thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions include those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C., all which are incorporated by reference in their entirety.

In some embodiments, the linking agent is a chelator. For example, the chelator comprising the molecule, DOTA (DOTA=1,4,7,10-tetrakis(carboxymethyl)-1,4,7,10-tetraazacyclododecane), that can readily be labeled with a radiolabel, such as Gd³⁺ and ⁶⁴Cu, resulting in Ge³⁺-DOTA and ⁶⁴Cu -DOTA respectively, attached to the quantum dot (nanoparticle). Optical properties of the cores (luminescence or fluorescence emission or plasmon frequency) are not affected by the addition of a silica shell or the presence of chelated paramagnetic ions. Other suitable chelates are known to those of skill in the art, for example, 1,4,7-triazacyclononane-N,N′,N″-triacetic acid (NOTA) derivatives being among the most well-known (see, e.g., Lee et al. (1997) Nucl Med Biol. 24:225-23019, incorporated by reference in its entirety).

In some embodiments the linking agent is a heterobifunctional crosslinker comprising two different reactive groups that form a heterocyclic ring that can interact with a peptide. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. In some embodiments, an affinity agent (also referred to as a specific binding pair), e.g., agents that specifically binds to a ligand, is the linking agent. For instance, a first linking agent is bound to the semiconductor nanocrystal (nanoparticle) and a second linking agent is bound to a reporter, targeting entity, imaging or therapeutic agent. Affinity agents include receptor-ligand pairs, antibody-antigen pairs and other binding partners such as streptavidin/avidin and biotin. In one illustrative embodiment, the first linking agent is streptavidin or avidin and the second linking agent is biotin. the streptavidin or avidin is bound to the nanoparticle and a biotinylated agent (e.g., biotinylated imaging agent, biotinylated therapeutic, biotinylated antibody, etc.) is conjugated to the nanoparticle via streptavidin/avidin-biotin linkage. In some embodiments, other biotinylated radiolabel, peptides, proteins, antibodies, dyes, probes and other small molecules are attached to the streptavidin or avidin, and thus the nanoparticle.

In another embodiment, pendent functionalized linkers such as natural or modified polysaccharides as well as natural and modified nucleic or amino acids can be useful.

### F. Libraries

A complex library of nanoparticle conjugates can be prepared by combining a plurality of nanoparticle conjugates of different types. As defined herein, a “library” refers to a collection of two or more different materials or types of materials, e.g., different nanoparticle conjugates. In one embodiment, each type of nanoparticle conjugate includes an oligonucleotide barcode and a targeting entity that are unique or different from other types of nanoparticle conjugates. By having unique oligonucleotide barcodes, the identity of the nanoparticle conjugate as well as information regarding the nanoparticle conjugate, e.g. identity of the targeting entity, reporter labels, size, polymer layers, etc., can be determined. In some embodiments, each type of nanoparticle conjugates is prepared separately then combined to form a library. The library can then be administering directly to the cells in vitro or in vivo. In other embodiments, each type of nanoparticle conjugate can be administered either separately or contemporaneously to the cells in vitro or in vivo. In one embodiment, the nanoparticles can be functionalized with any combination of targeting groups such as monoclonal antibodies, polyclonal antibodies, single chain antibodies, a bi-specific antibody, small molecules, peptides, peptoids, aptamers, and nanobodies. The composition and surface targeting group density of a single particle type can be varied to produce a library of different particle types. For example, particle type A is functionalized with antibody 1 and antibody 2 each at surface density X, particle type B is functionalized with antibody 1 and small molecule 3 at surface density Y, and so on. Particle libraries contain ideally 100-10,000 constituent members pooled together, but may contain between 2-100,000 if needed. The individual particle types can be stored separately in appropriate buffer (e.g. 1× PBS buffer, pH 7.4) to minimize cross-reactivity between particle types. See Weissleder et al. Nature Biotech 2005, Vol. 23(11), pp. 1418-1423, incorporated by reference in its entirety, for an example library comprised of 146 different types of nanoparticles.

### II. Methods for Characterizing a Cell

It is well-known that solid cancer tumors are comprised of genetically and phenotypically heterogeneous populations of cells due to a high rate of somatic mutation, clonal expansion, and diverse tumor microenvironment. This genetic and/or phenotypic diversity also manifests itself as cells are released from the primary tumor and begin to transit the circulatory system as circulating tumor cells (CTC). To characterize the genetic and phenotypic profile of tumor cells as well as obtain further information about the cells including surface markers, a complex library of tagged nanoparticle conjugates can be administered to cells in vitro or in vivo and the targeting efficiency and/or biodistribution properties can be evaluated by collecting tagged single cells and reading the tags to identify what particles are bound to the cell. The nucleic acids of the tagged cell are sequenced to provide further characterizing information concerning the cell's genotype and phenotype. Furthermore, the tags are sequenced to specifically determine the identity of the nanoparticle conjugates that bound to the cells. That is, it is possible to characterize, at the single cell level, not only the identity of the cell by its genotype and phenotype by profiling but also the identity of the specific nanoparticle conjugates (and therefore the cell surface markers) that are bound to the cell.

In one embodiment, a method is provided which entails exposing the tumor cells or a collection of CTCs to a complex library of nanoparticle conjugates of one or more types, wherein each type of nanoparticle conjugate has unique tag, followed by partitioning the tagged tumor cells, attaching partition specific adaptors to the nanoparticle conjugates and nucleic acid molecules of the tagged cells, attaching the common adaptors to the nucleic acid molecules of the tagged cells, amplifying the modified nucleic acid molecules, pooling the amplified library, preparing a sequencing library, and sequencing the library. By including the partition specific adaptor having a unique oligonucleotide barcode for each cell, multiple cell samples can be pooled together for a single sequencing run, thus enhancing the efficiency of the sequencing. Furthermore, by labeling the nucleic acids of the cell as well as the nanoparticle conjugates that bind to each cell with a unique partition specific adaptor, it is possible to identify and match the specific nanoparticle conjugates to the cell that it had bound to in the pooled mixture.

In one aspect, a method for characterizing a cell, e.g., a tumor cell, is provided. The method includes: (a) contacting cells with nanoparticle conjugates of one or more types to form labeled cells, each type of nanoparticle conjugate comprising a nanoparticle, targeting entities of one or more types, and tags of one or more types; (b) partitioning the labeled cells into separate partitions; (c) lysing each labeled cell to release nucleic acid molecules, each nucleic molecule having a first end and a second end; (d) adding partition specific adaptors and common adaptors to each partition, wherein each partition specific adaptor comprises an oligonucleotide barcode, wherein the partition specific adaptors are different for each partition, wherein each common adaptor comprises an oligonucleotide of a predetermined sequence and wherein the common adaptors are the same for each partition; (e) ligating the partition specific adaptor to a first end of each nucleic acid molecule and the common adaptor to a second end of each nucleic acid molecule to form modified nucleic acid molecules; (f) ligating the partition specific adaptor to the nanoparticle conjugate to form a partition specific adaptor modified nanoparticle conjugate; (g) amplifying the modified nucleic acid molecules and the partition specific adaptor of the partition specific adaptor modified nanoparticle conjugates; (h) pooling the amplified modified nucleic acid molecules and the amplified partition specific adaptors from the partition specific adaptor modified nanoparticle conjugates from each partition to form a library; and (i) sequencing the library. In one embodiment, the nucleic acid molecules comprise genomic DNA molecules and mRNA molecules. In another embodiment, prior to step (d), step (C1) is performed which entails reverse transcribing the mRNA molecules to form complementary DNA molecules (cDNA). In some embodiments, step (g) amplifying involves a polymerase chain reaction (PCR). In some embodiments, step (i) sequencing employs a massively parallel sequencer. In some embodiments, the separate partitions are contained in separate wells. In other embodiments, the tags of one or more types comprise oligonucleotide barcodes of one or more types.

In one embodiment, the method entails the isolation of single cells of interest from culture, tissue, dissociated cell suspensions, or blood, contacting the cells with a complex library of nanoparticle conjugates of one or more types, each type of nanoparticle having a unique tag such as an oligonucleotide barcode, to form a tagged cell, lysing the tagged cell to release its nucleic acid molecules comprising genomic DNA and mRNA, ligating the genomic DNA and mRNA (via conversion of mRNA into cDNA followed by cDNA amplification) with a partition specific adaptor and a common adaptor to form modified nucleic acid molecules, amplifying the modified nucleic acid molecules, pooling the amplified modified nucleic acid molecules from each partition to form a library, and sequencing of the library, e.g., massively parallel sequencing.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. Furthermore, in general, cells from any population can be used in the methods, including prokaryotic or eukaryotic single celled organisms such as bacteria or yeast. A single cell suspension can be obtained using standard methods known in the art including, for example, enzymatically using trypsin or papain to digest proteins connecting cells in tissue samples or releasing adherent cells in culture, mechanically separating cells in a sample, or isolation from a bodily fluid such as blood.

The single cells are then contacted with a nanoparticle conjugate library under suitable conditions such that the targeting entity of the nanoparticles will bind to markers present on the surface of the cell membrane to form tagged single cells. In a representative example, for in vitro experiments, single cells are incubated with 0.1 pg/mL-1.0 mg/mL nanoparticles for 1-24 hours at 37 C, with or without mixing, in the presence of 5% CO₂. For a reference of a bulk (non-single-cell) experiment, see Weissleder et al. Nature Biotech 2005, Vol. 23(11), pp. 1418-1423, incorporated by reference in its entirety.

The tagged single cells are then isolated. Methods for isolating single cells are known in the art and include a limiting dilution approach, a microfluidic single cell isolation platform such as Fluidigm C1, a bulk cell sorter (CD FACSAria), fluorescence activated cell sorting (FACS), micromanipulation, optical tweezers, the use of semi-automated cell pickers (e.g. the QUIXELL cell transfer system from Stoelting Co.), or a combination of these approaches. Rare tumor cells such as CTCs which a few are present among millions of blood cells can be isolated from patients using epithelial cell surface markers and microfluidic-based technologies. See, for instance, Saliba et al., Nucleic Acid Res., published Jul. 22, 2014 (doi: 10.1093/nar/gku555), incorporated by reference in its entirety. Individual cells can, for example, be individually selected based on features detectable by microscopic observation, such as location, morphology, or reporter gene expression.

The isolated tagged cells are then individually partitioned into any suitable reaction support or vessel in which the single cells can be treated individually. For instance, a 96-well titer plate can be used where each tagged single cell is placed in a single well. Alternatively, the single cells can be placed via droplets or any suitable manner onto spatially discrete areas of a substrate such as a glass slide. The support can have any suitable immobilization layer such as a polymer matrix. Each partitioned cell is then lysed to release nucleic acid molecules, e.g., genomic DNA and mRNA of the single cell transcriptome. Any suitable cell lysing method can be used. Lysis can be achieved by, for example, heating the cells, or by the use of detergents or other chemical methods, or by a combination of these. In some instances, a mild lysis procedure can advantageously be used to prevent the release of nuclear chromatin, thereby avoiding genomic contamination of the cDNA library, and to minimize degradation of mRNA. For example, heating the cells at 72° C. for 2 minutes in the presence of Tween-20 is sufficient to lyse the cells while resulting in no detectable genomic contamination from nuclear chromatin. Alternatively, cells can be heated to 65° C. for 10 minutes in water (Esumi et al., Neurosci Res 60(4):439-51 (2008))(incorporated by reference in its entirety); or 70° C. for 90 seconds in PCR buffer II (Applied Biosystems) supplemented with 0.5% NP-40 (Kurimoto et al., Nucleic Acids Res 34(5):e42 (2006))(incorporated by reference in its entirety); or lysis can be achieved with a protease such as Proteinase K or by the use of chaotropic salts such as guanidine isothiocyanate (U.S. Publication No. 2007/0281313) (incorporated by reference in its entirety). The lysing solution can be added by any suitable means such as droplets or pipetting the solution. The tagged single cells can then be subject to amplification and sequencing methods to characterize the cell genotype and/or phenotype via a variety of suitable techniques such as whole genome sequencing, RNAseq, exome capture, and mass spectrometry; and (ii) the identity of the oligonucleotide barcode and thus the identity of nanoparticle conjugate bound to the single cell.

If single cell profiling involving mRNA sequencing will be performed in addition to or instead of single cell profiling of genomic DNA, then reverse transcription of mRNA is generally required as it is not yet possible to directly sequence RNA molecules. Profiling of genomic DNA and mRNA can be done separately or together.

Synthesis of cDNA from mRNA in the methods described herein can be performed directly on cell lysates, such that a reaction mix for reverse transcription is added directly to cell lysates. Alternatively, mRNA can be purified after its release from cells. This can help to reduce mitochondrial and ribosomal contamination. The mRNA purification can be achieved by any method known in the art, for example, by binding the mRNA to a solid phase. Commonly used purification methods include paramagnetic beads (e.g. Dynabeads). Alternatively, specific contaminants, such as ribosomal RNA can be selectively removed using affinity purification. Methods for synthesizing cDNA from small amounts of mRNA, including from single cells, have previously been described (Kurimoto et al., Nucleic Acids Res 34(5):e42 (2006): Kurimoto et al., Nat Protoc 2(3):739-52 (2007); and Esumi et al., Neurosci Res 60(4):439-51 (2008)), all incorporated by reference in their entirety).

In order to generate an amplifiable cDNA, these methods introduce a primer annealing sequence at both ends of each cDNA molecule in such a way that the cDNA library can be amplified using a single primer. The Kurimoto method uses a polymerase to add a 3′ poly-A tail to the cDNA strand, which can then be amplified using a universal oligo-T primer. In contrast, the Esumi method uses a template switching method to introduce an arbitrary sequence at the 3′ end of the cDNA, which is designed to be reverse complementary to the 3′ tail of the cDNA synthesis primer. Again, the cDNA library can be amplified by a single PCR primer. Single-primer PCR exploits the PCR suppression effect to reduce the amplification of short contaminating amplicons and primer-dimers (Dai et al., J Biotechnol 128(3):435-43 (2007), incorporated by reference in its entirety). As the two ends of each amplicon are complementary, short amplicons will form stable hairpins, which are poor templates for PCR. This reduces the amount of truncated cDNA and improves the yield of longer cDNA molecules.

For single cell whole genome analysis, following cell lysis, the first step is typically whole genome amplification (WGA, see Bourcy et al. PLOS One, 2014, Vol. 9(8), e105585, pp. 1-9); and Zong et al. Science 2012, Vol. 338, p. 1622-1626 (both incorporated by reference in their entirety); PicoPLEX WGA Kit from Rubicon Genomics; and REPLI-g single cell kit from QIAGEN, GenomePlex WGA kit from Sigma, etc.). After WGA, the amplified molecules are can be used as-is or be cut into smaller fragments using a transposase (see, for instance, Nextera sample prep kit from Illumina), sheared using adaptive focused acoustics (Covaris), or cut enzymatically. In the case of Nextera kit, the transposase adds the adaptors simultaneously with the fragmentation step. See Illumina website for details at http://www.illumina.com/products/nextera_dna_sample_prep_kit.html, which is incorporated by reference in its entirety.

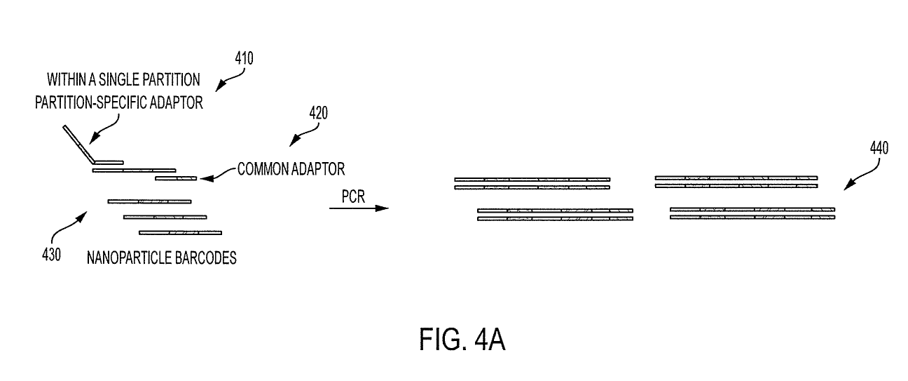

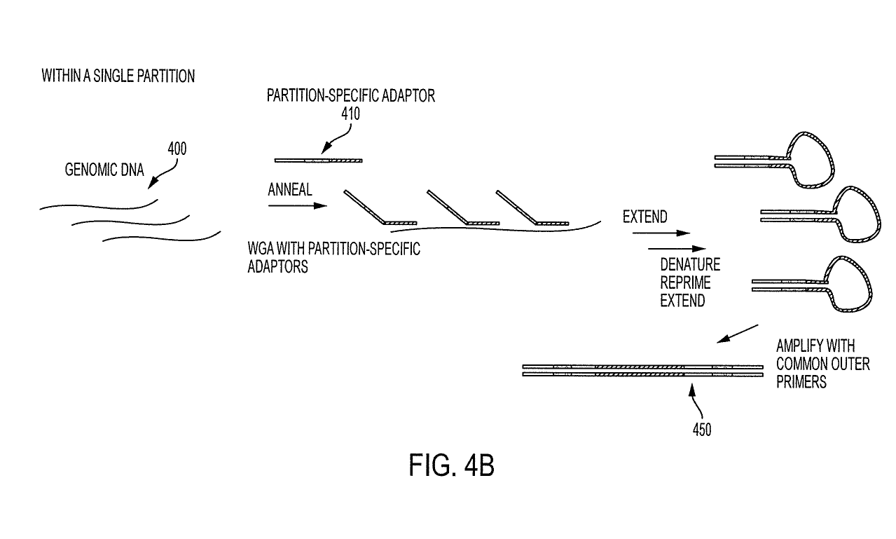

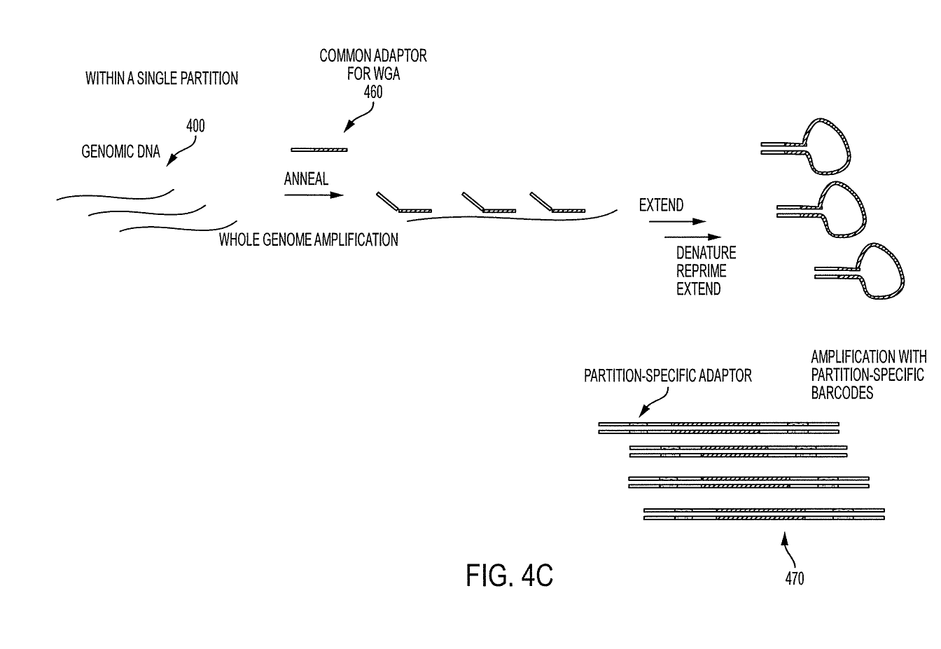

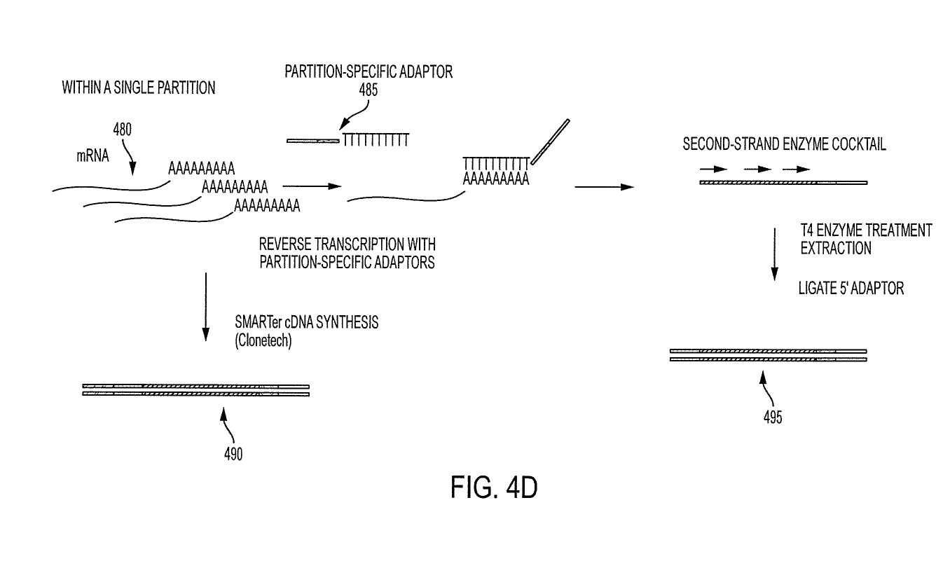

In one embodiment, partition specific adaptors and common adaptors are added to each lysed cell in each partition and are ligated to the nucleic acids. The partition specific and common adapters can be added in any order or simultaneously as desired. As defined herein, the phrase “partition specific adaptor” refers to an oligonucleotide adaptor having a barcode that is unique for each partitioned cell. The partition specific adaptors can be PCR primers designed to specifically amplify the nanoparticle barcodes, and can range in size from 20-100 nucleotides. They can also be incorporated into primers designed to amplify the genome or transcriptome (see for instance, FIGS. 4A to 4D. That is, the unique barcode can associate the nucleic acid molecules with a particular single partitioned cell as well as a nanoparticle conjugate, even after the nucleic acid samples are pooled together as described below The partition specific adaptor (tag) can serve a number of purposes including (a) matching nanoparticle tags to a cell's genomic DNA/RNA molecules, and (b) serve as a quality control metric. For example, certain partition-specific tags can be associated with certain cell or nanoparticle types, and if it was discovered that partitions that contain cells or nanoparticles that should not be present, the partition specific adaptor will act as an indicator of contamination. The phrase “common adaptor” refers to a an oligonucleotide sequence that does not contain a uniquely identifying sequence. Common adaptor sequences can be shared across partitions and are generally used to assist with amplification. See FIG. 4 for clarifying information. For nanoparticle tag amplification as shown in FIG. 4A, one can use a partition specific adaptor (PCR primer) 410 and a common adaptor (PCR primer) 420 to perform PCR amplication of nanoparticle barcodes 430 to produce duplex DNA molecules 440. For genomic DNA amplification as shown in FIG. 4B of genomic DNA 400, the whole genome amplification (WGA) step can proceed with a partition-specific primer 410 (using established methods such as PicoPLEX, REPLI-G, etc.) followed by amplification with common primers (not shown) to produce amplified genomic DNA 450. Alternatively, whole genome amplification (WGA) of genomic DNA 400 as shown in FIG. 4C can proceed with a common set of primers 460 and partition-specific primers can be added during a subsequent round of amplification to produced amplified genomic DNA 470. Finally, FIG. 4D illustrates two paths for amplification of mRNA 480 via reverse transcription with partition specific adaptors 485 to produce cDNA molecules 490 and 495.

All the partitioned cells will have the same common adaptor. The partition specific adaptors are covalently bound or ligated to a first end of the nucleic acid molecules while the common adaptors are covalently bound or ligated to the second end of the nucleic acid molecules. Generally, either ends of the particle tags and genomic DNA while the 3′ end of cDNA molecules can be used. The partition specific adaptors are also covalently bound to the oligonucleotides of the nanoparticle conjugate(s) in each partition. For the nanoparticle barcodes, primers specific for flanking common adaptor sequence can be designed to incorporate partition-specific barcodes. For genomic DNA, the partition specific adaptors can be incorporated into the primers used during initial WGA or at a subsequent amplification step. For RNA, the partition specific adaptors can be added during the reverse transcription (cDNA generation) step.

The partition/common adaptor modified nucleic acid molecules and the partition specific adaptor modified nanoparticle conjugates are then amplified by any suitable means. For whole genomic amplification (WGS), a variety of methods can be used including, multiple displacement amplification (MDA) and MALBEC (see Zong et al. Science 2012, Vol. 338, p. 1622-1626, which is incorporated by reference in its entirety). Commercial kits for amplification of DNA molecules via MDA include, without limitation, GenomePlex by Sigma-Aldrich, REPLI-q by Qiagen, GenomiPhys by Amersham, RepliPHI by Epicentre). For amplifying mRNA, reverse transcription is performed to produced cDNA followed by cDNA amplification as described above. Commercial kits for amplifying mRNA are available Clontech SMARTer PCR cDNA synthesis kit, Cells-to-cDNA kit II (Life Technologies), ProtoScript First Strand cDNA synthesis kit (New England Biolabs).

The amplified modified nucleic acid molecules and the amplified partition specific adaptors from the modified nanoparticle conjugates from each partition are then pooled to form a library. This pooled library can be used for sequencing or can be further processed to obtain a library suitable for massively parallel sequencing. As defined herein, “massively parallel sequencing” or “massive parallel sequencing” or “next-generation sequencing” refers to any high-throughput approach to DNA sequencing. As used herein, a library is suitable for sequencing when the complexity, size, purity or the like of a genomic and/or cDNA library is suitable for the desired screening method. In particular, the library can be processed to make the sample suitable for any high-throughput screening methods, such as Applied Biosystems' SOLiD sequencing technology, or Illumina's Genome Analyzer. If desired, the pooled library can be further amplified, e.g. by PCR, to obtain a sufficient quantity of DNA for sequencing. In one embodiment, the library can be sequenced to provide an analysis of gene expression in a plurality of single cells. As defined herein, the term “gene” refers to a poly nucleotide containing at least one open reading frame (ORF) that is capable of encoding a particular polypeptide or protein after being transcribed and translated. Any of the polynucleotide sequences described herein can be used to identify larger fragments or full-length coding sequences of the gene with which they are associated. Methods of isolating larger fragment sequences are known to those of skill in the art. As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

The library can be sequenced by any suitable high-throughput screening method. In particular, the cDNA library can be sequenced using a high-throughput screening method, such as Applied Biosystems' SOLiD sequencing technology, or Illumina's Genome Analyzer. In one aspect of the present disclosure, the cDNA library can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. As defined herein, a “read” is a length of continuous nucleic acid sequence obtained by a sequencing reaction. As defined herein, “Shotgun sequencing” refers to a method used to sequence very large amount of DNA (such as the entire genome). Methods and programs for shotgun sequencing a cDNA library are well known in the art.

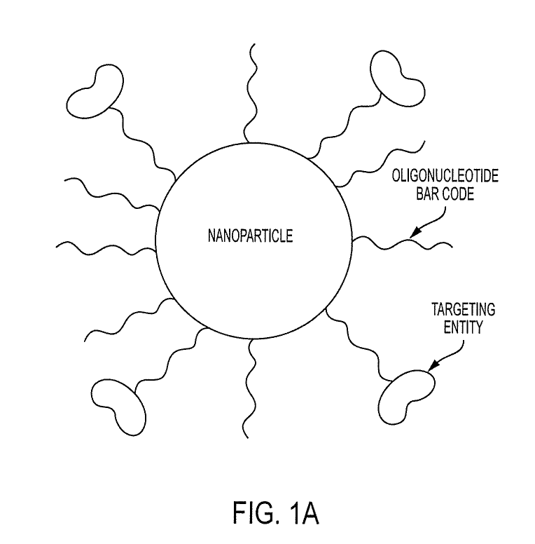

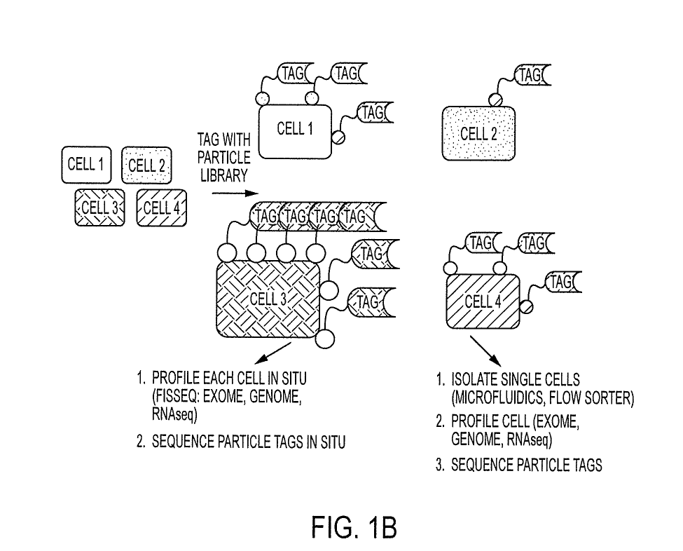

A method of the present disclosure is illustrated in FIG. 1B. Single cells in the form of a suspension are obtained from tumor tissue of interest. The cell suspension is combined with a library of nanoparticle conjugates of one or more types, each type of nanoparticle conjugate having a unique oligonucleotide barcode (a tag), under suitable conditions to allow the targeting entity of the nanoparticle conjugates to bind to its target, e.g., marker, on the cell surface to form a tagged cell. The tagged single cells can then be profiled by any suitable method including, for example, fluorescent in situ sequencing (FISSEQ) which is a method of sequencing DNA by the use of polonies and cycles of fluorescent deoxyribonucleotide triphosphates (dNTP); RNA sequencing (RNA-seq) which is also referred to as Whole Transcriptome Shotgun Sequencing (WTSS) and employs next-generation sequencing; exome sequencing; and genome sequencing. The oligonucleotide barcodes from the nanoparticle conjugates are also sequenced to obtain the identity of the nanoparticle conjugate and to match the nanoparticle conjugate to a specific cell.

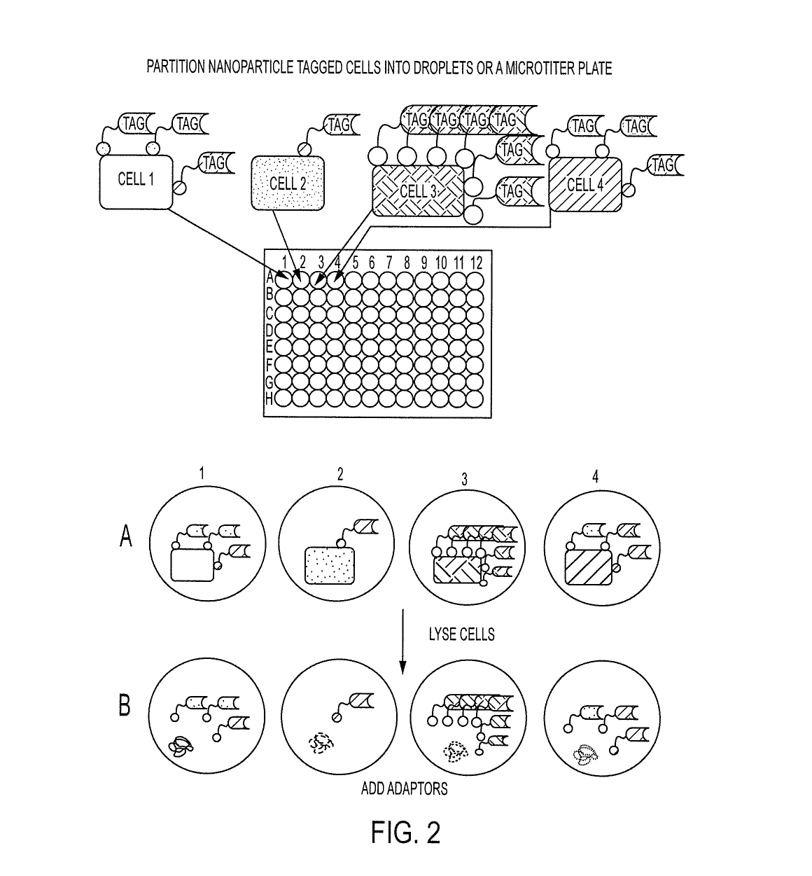

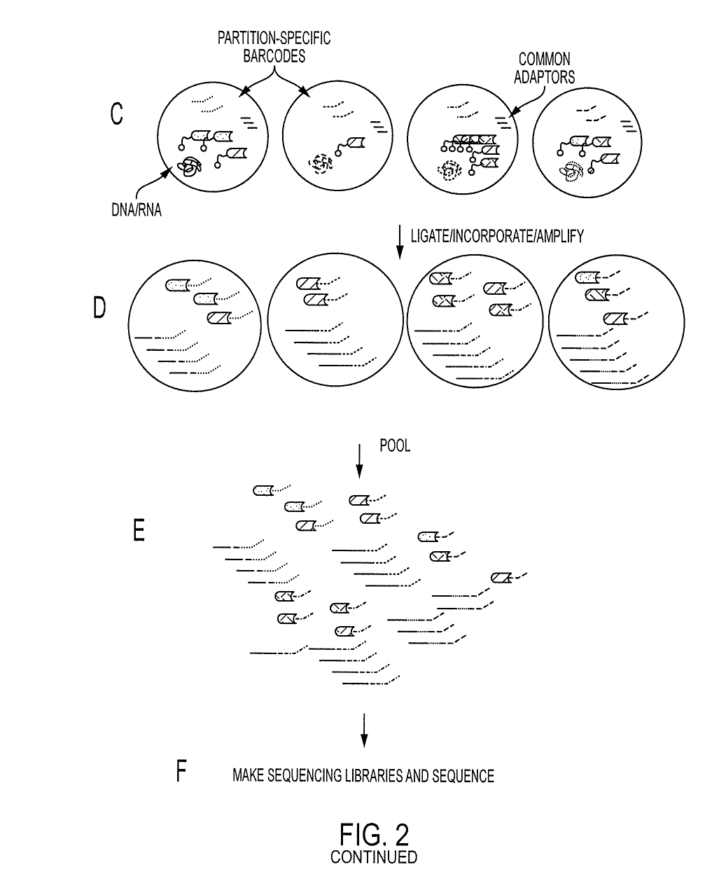

FIG. 2 illustrates a representative method of the present disclosure. In FIG. 2A, single tagged cells are placed in separate wells of a 96-well plate. In FIG. 2B, lysing solution is added to each well to release the cellular nucleic acid content. If desired, a reverse transcription reaction mix can be added directly to the lysates without additional purification. This results in the synthesis of cDNA from cellular mRNA and allows for subsequent amplification and sequencing of both genomic DNA and mRNA. In FIG. 2C, partition specific adaptors and common adaptors are added to each well. The adaptors can be added in any order or together. The adaptors are then ligated to the nucleic acid molecules to form modified nucleic molecules. The modified nucleic acid molecules in each well are then amplified (FIG. 2D), and the amplified molecules of all the wells are pooled (FIG. 2E). In FIG. 2F, the pooled mixture is then further modified to make suitable sequencing libraries and then sequenced by next-generation sequencing techniques. In general, the sequencing process produces around 100 million reads which allows for identification of genes that are expressed in each single cell and/or gene variants. See Streets et al. PNAS 2014, Vol. 111(19), pp. 7048-7053; Wu et al. Nature Methods 2014, Vol 11(1), pp. 41-46 and online methods doi:10.1038/nmeth.2694; Patel et al. Science 2014, Vol. 344 (issue 6190), pp. 1396-1401, all which are incorporated by reference in its entirety.

In another aspect, another method for characterizing a cell is provided. The method involves (a) contacting cells with nanoparticle conjugates of one or more types to form labeled cells, each type of nanoparticle conjugate comprising a nanoparticle, targeting entities of one or more types, and a plurality of first oligonucleotide barcodes; (b) partitioning and immobilizing the labeled cells into spatially discrete regions on a support, each labeled cell comprising mRNA molecules; (c) sequencing the first oligonucleotide barcodes of each labeled cell; (d) reverse transcribing the mRNA molecules of each labeled cell into cDNA molecules to form a library for each labeled cell; and (e) sequencing the library for each labeled cell. In one embodiment, steps (c) and (e) sequencing involves fluorescent in situ sequencing. In other embodiment, the support comprises a glass slide coated with a gel matrix.

The preparation and isolation of single cells as well as labeling or tagging the single cells with nanoparticle conjugates and partitioning the tagged cells are described above. The tagged cells are partitioned and immobilized in spatially discrete regions onto the surface of a support, e.g., microscope slides, composed of any suitable material such as glass. Examples of suitable supports include microscope slides, glass coverslips, and microwell flat bottom plates. The supports can include a polymer gel matrix for immobilizing the cells. See Lee et al, Nature Protocols, 2015, Vol. 10(3), pp. 442-458, incorporated by reference in its entirety, for detailed methods.

The oligonucleotide barcodes of the nanoparticle conjugate for each partitioned cell are then sequenced in situ by any suitable method such as FISSEQ. See, for instance, Lee et al. Nature Protocols, 2015, Vol. 10(3), pp. 442-458, Lee et al. Science 2014, Vol. 343, pp. 1360-1363; Raj et al. Nature Methods 2008, Vol. 5(10), pp. 877-879; Lubeck et al. Nature Methods, April 2014, Vol. 11(4), pp. 360-361; Choi et al. Nature Biotech 2010, Vol. 28(11), pp. 1208-1212; Ke et al. Nature Methods 2013, Vol. 10(9), pp. 857-860 and online method Doi: 10.1038/nmeth.2563), all which are incorporated by reference in its entirety. In one representative method, fluorescent in situ sequencing method of the oligonucleotide barcodes are performed.

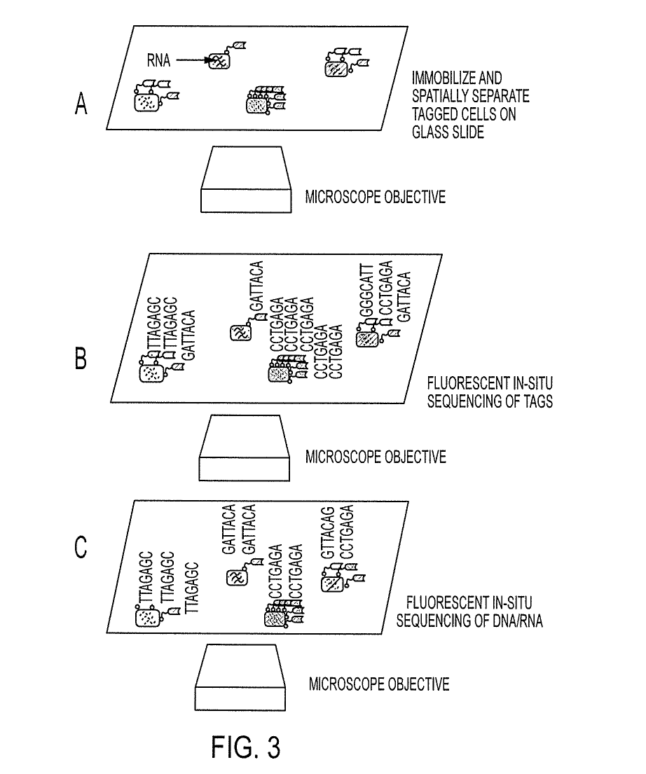

Following oligonucleotide barcode sequencing, the genomic DNA and RNA of each single cell is then amplified and sequenced by any suitable method as described above. FIG. 3 illustrates an example method for characterizing cells. Single cells are contacted with a library of nanoparticle conjugates of several types to form tagged cells which are bound to one or more types of nanoparticle conjugates, each type of nanoparticle conjugates having a unique oligonucleotide barcode or tag. The nanoparticle conjugates include targeting entities that are specific to cell surface markers. Each tagged cell is then partitioned by immobilization onto spatially discrete areas on a glass microscope slide (FIG. 3A). The tags of the nanoparticle conjugates of each partition are separately sequenced (FIG. 3B). The nucleic acid content of each partitioned cell is separately sequenced (FIG. 3C).

### II. CONCLUSION

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A method comprising:
(a) contacting cells with nanoparticle conjugates of one or more types to form labeled cells, each type of nanoparticle conjugate comprising a nanoparticle, targeting entities of one or more types, and tags of one or more types;
(b) partitioning the labeled cells into separate partitions;
(c) lysing each labeled cell to release nucleic acid molecules, each nucleic molecule having a first end and a second end;
(d) adding partition specific adaptors and common adaptors to each partition, wherein each partition specific adaptor comprises an oligonucleotide barcode, wherein the partition specific adaptors are different for each partition, wherein each common adaptor comprises an oligonucleotide of a predetermined sequence and wherein the common adaptors are the same for each partition;
(e) ligating the partition specific adaptor to a first end of each nucleic acid molecule and the common adaptor to a second end of each nucleic acid molecule to form modified nucleic acid molecules;
(f) ligating the partition specific adaptor to the nanoparticle conjugate to form a partition specific adaptor modified nanoparticle conjugate;
(g) amplifying the modified nucleic acid molecules and the partition specific adaptor of the partition specific adaptor modified nanoparticle conjugates;
(h) pooling the amplified modified nucleic acid molecules and the amplified partition specific adaptors from the partition specific adaptor modified nanoparticle conjugates from each partition to form a library; and
(i) sequencing the library.

2. The method according to claim 1, wherein the nucleic acid molecules comprise genomic DNA molecules and mRNA molecules.

3. The method according to claim 2, wherein prior to step (d), further comprising step (C1) reverse transcribing the mRNA molecules to form complementary DNA molecules (cDNA).

4. The method according to claim 1, wherein step (g) amplifying involves a polymerase chain reaction (PCR).

5. The method according to claim 1, wherein step (i) sequencing employs a massively parallel sequencer.

6. The method according to claim 1, wherein the separate partitions are contained in separate wells.

7. The method according to claim 1, wherein the tags of one or more types comprises an oligonucleotide barcodes of one or more types.

8. A method comprising:
(a) contacting cells with nanoparticle conjugates of one or more types to form labeled cells, each type of nanoparticle conjugate comprising a nanoparticle, targeting entities of one or more types, and a plurality of first oligonucleotide barcodes;
(b) partitioning and immobilizing the labeled cells into spatially discrete regions on a support, each labeled cell comprising mRNA molecules;
(c) sequencing the first oligonucleotide barcodes of each labeled cell;
(d) reverse transcribing the mRNA molecules of each labeled cell into cDNA molecules to form a library for each labeled cell; and
(e) sequencing the library for each labeled cell.

9. The method according to claim 8, wherein steps (c) and (e) sequencing involves fluorescent in situ sequencing.

10. The method according to claim 8, wherein the support comprises a glass slide coated with a gel matrix.
